(19) United States
(12) Patent Application Publication
(10) Pub. No.: US 2007/0112261 A1
(43) Pub. Date: May 17, 2007

(54) Automobile Glucose Sensor Monitoring System and Method for Using the Same

(75) Inventors: Bradley J. Enegren, Moorpark, CA (US); David Kerr, Dorset (GB)

Correspondence Address: MEDTRONIC MINIMED INC., 18000 DEVONSHIRE STREET, NORTHRIDGE, CA, 91325-1219, US

(73) Assignee: MEDTRONIC MINIMED, INC., Northridge, CA (US)

(21) Appl. No.: 11/466,532

(22) Filed: Aug. 23, 2006

Related U.S. Application Data
(60) Provisional application No. 60/711,167, filed on Aug. 24, 2005.

(51) Int. Cl. A61B 5/00

(52) U.S. Cl. 600/365

(57) ABSTRACT

An automobile monitoring system is for monitoring patient body characteristics. The automobile monitoring system includes at least one sensor to monitor at least one patient body characteristic, at least one transmitter operatively coupled to the at least one sensor to communicate sensor data, at least one monitor operatively coupled to the at least one transmitter to receive the sensor data, and automobile electronics operatively coupled to the at least one transmitter to receive sensor data. The at least one monitor and the automobile electronics display the sensor data to the patient. The at least one transmitter communicates with the at least one monitor and the automobile electronics using at least one wireless protocol including Bluetooth, infrared, radio frequency, 802.11a, 802.11b, or 802.11g. The automobile electronics include at least one of a GPS navigation system, a DVD entertainment system, an on-system computer, or a stereo system.

### RELATED APPLICATIONS

This application claims the benefit of prior filed U.S. Provisional Application Ser. No. 60/711,167, filed on Aug. 24, 2005.

### FIELD OF THE INVENTION

Embodiments of the invention relate to improved sensor monitoring systems and, more particularly, to devices and methods for connecting a glucose sensor monitoring system to automobile electronics.

### BACKGROUND OF THE INVENTION

Diabetes is a disease in which the body does not produce or properly use insulin. Approximately 13 million people in the United States have been diagnosed with some form of diabetes. Type 1 diabetes results from the body's failure to produce insulin. Type 2 diabetes results from insulin resistance in which the body fails to properly use insulin. In order to effectively manage the disease, diabetics must closely monitor and manage their blood glucose levels through exercise, diet and medications. In particular, both Type 1 and Type 2 diabetics rely on insulin delivery and blood glucose monitoring to control their diabetes.

External infusion devices have been used to deliver medication to a patient as generally described in U.S. Pat. Nos. 6,554,798 and 6,551,276 which are specifically incorporated by reference herein. In addition to delivering medication to a patient, other medical devices have been used to determine body characteristics by obtaining a sample of bodily fluid. A variety of implantable electrochemical sensors have been developed for detecting and/or quantifying specific agents or compositions in a patient's blood. For instance, glucose sensors have been developed for use in obtaining an indication of blood glucose levels in a diabetic patient. Such readings can be especially useful in monitoring and/or adjusting a treatment regimen that typically includes the regular administration of insulin to the patient. Thus, blood glucose readings are particularly useful in improving medical therapies with semi-automated medication infusion pumps of the external type and/or implantable type.

Monitoring blood glucose levels plays an integral role in the management and control of diabetes. Finger stick measurements, glucose sensors and monitors have traditionally been used to check the blood glucose levels of diabetic patients. In recent years, continuous glucose monitoring systems have been developed utilizing the latest sensor technologies incorporating both implantable and external sensors as generally described in U.S. Pat. No. 5,391,250 entitled “Method of Fabricating Thin Film Sensors”, U.S. Pat. No. 6,484,046 entitled “Electrochemical Analyte Sensor,” and U.S. Pat. Nos. 5,390,671, 5,568,806 and 5,586,553, entitled “Transcutaneous Sensor Insertion Set,” all of which are specifically incorporated by reference herein. Newer systems deliver the preciseness of finger stick measurements coupled with the convenience of not having to repeatedly prick the skin to obtain glucose measurements. These newer systems provide the equivalent of over 200 finger stick readings per day. Additionally, continuous glucose monitoring systems allow physicians and patients to monitor blood glucose trends of their body and suggest and deliver insulin based on each patient's particular needs. Accordingly, physicians and medical device companies are always searching for more convenient ways to keep diabetic patients aware of their blood glucose levels throughout the day.

### SUMMARY OF THE DISCLOSURE

According to an embodiment of the invention, an automobile monitoring system is for monitoring patient body characteristics. The automobile monitoring system includes at least one sensor to monitor at least one patient body characteristic, at least one transmitter operatively coupled to the at least one sensor to communicate sensor data, at least one monitor operatively coupled to the at least one transmitter to receive the sensor data, and automobile electronics operatively coupled to the at least one transmitter to receive sensor data. In alternative embodiments, the at least one monitor and the automobile electronics display the sensor data to the patient. In still further embodiments, the at least one patient body characteristic is blood glucose. In still additional embodiments, the at least one transmitter communicates with the at least one monitor and the automobile electronics using at least one wireless protocol. In particular embodiments, the at least one wireless protocol includes Bluetooth, infrared, radio frequency, 802.11a, 802.11b, or 802.11g. In other embodiments, the automobile electronics include at least one of a GPS navigation system, a DVD entertainment system, an on-system computer, or a stereo system.

According to further embodiments of the invention, the automobile electronics include default maximum and minimum thresholds for the at least one patient body characteristic. In alternative embodiments, the automobile electronics prevent ignition of the automobile when the sensor data is above the maximum threshold or below the minimum threshold. In still further embodiments, the automobile electronics display at least one warning when the sensor data is above the maximum threshold or below the minimum threshold. In yet additional embodiments, the automobile electronics sound at least one alarm when the sensor data is above the maximum threshold or below the minimum threshold. In other embodiments, the at least one alarm provides at least one of audio, visual or tactile indications.

According to yet another embodiment of the invention, a method for monitoring patient body characteristics in an automobile is disclosed. The method first installs at least one sensor in the body of a patient to monitor at least one patient body characteristic. The sensor is coupled to at least one transmitter to communicate sensor data. Next, sensor data is sent to at least one monitor coupled to the at least one transmitter. Sensor data is also sent to automobile electronics operatively coupled to the at least one transmitter. The sensor data is then displayed on the at least one monitor and the automobile electronics. In further embodiments, the at least one patient body characteristic is blood glucose. In still additional embodiments, the at least one transmitter communicates with the at least one monitor and the automobile electronics using at least one wireless protocol. In particular embodiments, the at least one wireless protocol includes Bluetooth, infrared, radio frequency, 802.11a, 802.11b, or 802.11g. In other embodiments, the automobile electronics includes at least one of a GPS navigation system, a DVD entertainment system, an on-system computer, or a stereo system.

Other features and advantages of the invention will become apparent from the following detailed description, taken in conjunction with the accompanying drawings which illustrate, by way of example, various features of embodiments of the invention.

### BRIEF DESCRIPTION OF THE DRAWINGS

A detailed description of embodiments of the invention will be made with reference to the accompanying drawings, where like numerals designate corresponding parts or cross-sections in the several figures.

FIG. 1 shows an embodiment of the invention utilizing a continuous glucose monitoring system including a sensor, transmitter and monitor.

FIG. 2 shows an embodiment of the invention utilizing the glucose monitoring system of FIG. 1 with an automobile vehicle.

FIG. 3 shows a block diagram an embodiment of the invention.

FIG. 4 shows a bock diagram of a further embodiment of the invention.

### DETAILED DESCRIPTION OF THE PREFERRED EMBODIMENTS

As shown in the drawings for purposes of illustration, the invention is embodied in a glucose monitoring system for use with an automobile vehicle. In particular embodiments of the invention, a real-time continuous glucose monitoring system communicates with electronics of an automobile to display real-time glucose sensor measurements and provide information related to high and low blood glucose levels to the patient in addition to blood glucose related trends using graphs and other analytical models.

The sensor included in the automobile glucose monitoring system may be inserted in and/or through subcutaneous, dermal, sub-dermal, inter-peritoneal or peritoneal tissue. In other embodiments of the invention, the sensor may be coupled to a monitor for determining glucose levels in the blood and/or body fluids of the patient without the use of, or necessity of, a wire or cable connection between the transmitter and the monitor. In these embodiments, the sensor utilizes glucose oxidase to determine glucose levels. In still further embodiments, the sensor may use other materials such as optical, fluorescence or electrical materials to determine glucose levels. It will be recognized that further embodiments of the invention may be used to determine the levels of other agents, characteristics or compositions, such as hormones, cholesterol, medication concentrations, pH, oxygen saturation, viral loads (e.g., HIV), or the like. In other embodiments, the sensor may also include the capability to be programmed or calibrated using data received by a telemetered characteristic monitor transmitter device, or may be calibrated at the monitor device (or receiver), as described in U.S. Pat. No. 6,809,653 entitled “Telemetered Characteristic Monitor System And Method Of Using The Same,” which is specifically incorporated by reference herein. The telemetered characteristic monitor system may be primarily adapted for use in subcutaneous human tissue. However, still further embodiments may be placed in other types of tissue, such as muscle, lymph, organ tissue, veins, arteries or the like, and used in animal tissue. Embodiments may provide sensor readings on an intermittent, near-continuous and/or continuous basis.

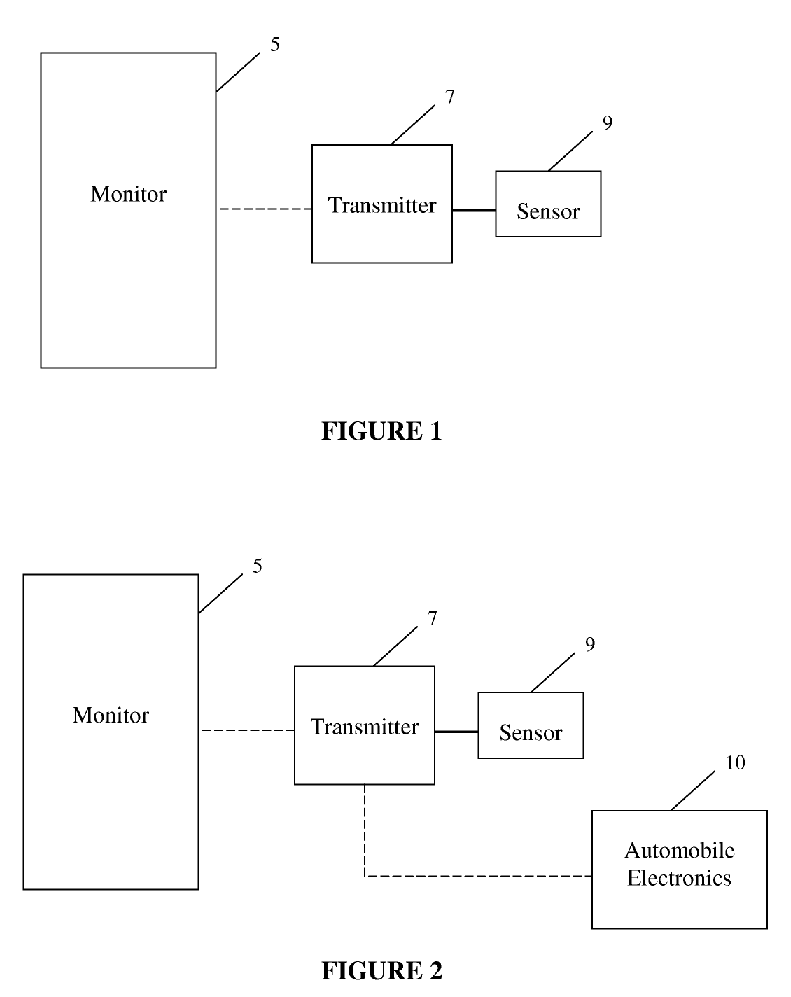

In particular embodiments of the invention, a glucose monitoring system, as shown in FIG. 1, displays real-time glucose values to the patient. In these embodiments, the glucose monitoring system includes a sensor 9 for measuring an agent such as blood glucose levels and the like. The sensor 9 may be connected by a wire to a transmitter 7. In other embodiments, the sensor and transmitter may be integrated into one unit or the sensor may have a built-in transmitter. The transmitter 7 provides the necessary electronics to communicate the sensor data to the glucose monitor 5. In still further embodiments, the transmitter attached to the sensor may also serve as a receiver to receive data from the monitor, a computer, an external infusion device or the like.

The monitor may include an LCD to display the sensor data. In other embodiments, the monitor may include an alarm and/or multiple alarms that activate when high and/or low blood glucose levels are detected. These alarms may be in the form of audible, visual, and/or tactile indications. In other embodiments, the alarms may activate upon user programmed instances such as an abnormal highs and/or lows in glucose levels for a particular time of the day.

In other embodiments, the glucose monitoring system may be adapted to communicate to automobile electronics. In particular embodiments, as shown in FIG. 2, the sensor 9 and transmitter 7 communicate directly with automobile electronics 10 including GPS navigation system, DVD entertainment system, on-system computer, stereo system or the like. In these embodiments, the automobile electronics may function as the traditional glucose monitor and display the sensor data to the patient on the dashboard, LCD screen located in the automobile, GPS navigation screen, DVD screen, stereo screen or the like. In additional embodiments, the automobile electronics may include algorithms to display sensor derived graphs and/or charts based on the patient's sensor data.

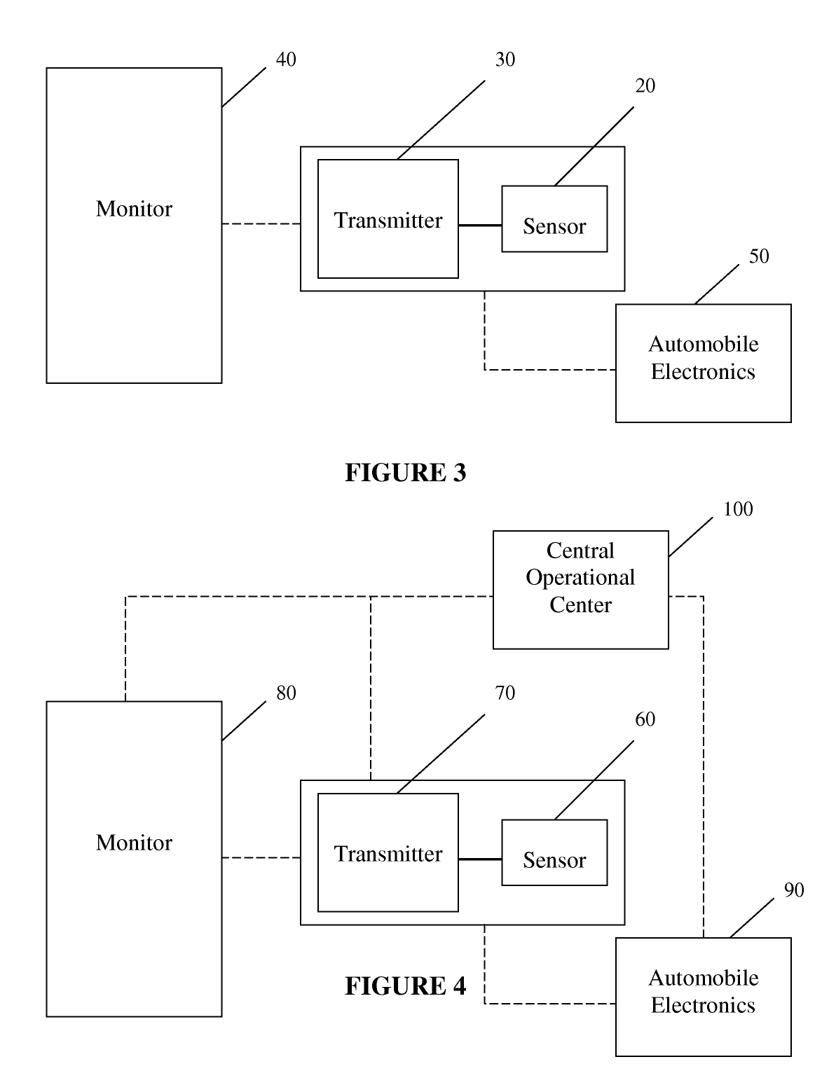

In further embodiments, as shown in FIG. 3, the glucose sensor 20 and transmitter 30 may communicate directly with the glucose monitor 40 and/or the automobile electronics 50 as described above. In particular embodiments, the transmitter may communicate using wireless protocols such as Bluetooth, Infrared, Radio Frequency, 802.11a, 802.11b, 802.11g, or the like. The transmitter may be equipped to handle multiple communication protocols and/or a single communication protocol. In still additional embodiments, the transmitter may communicate with the glucose monitor and or automobile electronics via a wired connection. The wire may either run from the transmitter into the glucose monitor and/or a port installed in the automobile's dashboard, GPS navigation system, DVD entertainment system, on-system computer, stereo system or the like. The wired port may use a standard computer connector port including serial, parallel, USB, firewire (IEEE 1394), or the like.

In additional embodiments, the glucose monitoring system may connect to the automobile electronics using any of the communication protocols described above. In these embodiments, the sensor and transmitter may connect the moment the patient unlocks the automobile door, enters the automobile, places the key in the ignition or the like. Upon connection, algorithms may be in place that allow the automobile electronics to prevent ignition of the automobile if sensor data indicates glucose levels above and/or below particular threshold values. Alternatively, in other embodiments, a warning may be displayed to the patient on the monitor itself and/or the automobile electronics notifying the user of high and/or low values.

In other embodiments, the automobile electronics may provide indications, data, graphs and/or trends on the dashboard, LCD screen located in the automobile, GPS navigation screen, DVD screen, stereo screen or the like. In alternative embodiments, the automobile electronics may provide alarms based on factory and/or user specified occurrences. Examples include high blood glucose levels, low blood glucose levels and the like. The alarms may come in the form of audio, visual, and/or tactile indications.

In other embodiments, the glucose monitoring system may provide warnings and/or alarms to the patient while the patient is operating the automobile. These warnings and/or alarms may be based on high blood glucose readings, low blood glucose readings, or the like. In the event a warning and/or alarm is displayed to the patient while the automobile is in operation, the patient may be prompted to pull over the automobile and check the sensor readings prior to continued operation of the automobile. The safety factors associated with such a system provide diabetic patients and the public with advanced warnings of potential hypo- and/or hyper-glycemic situations.

In still further alternative embodiments, as shown in FIG. 4, automobiles equipped with On-Star® technology and/or any other similar communication protocols may transmit the sensor data to a central operational center. In particular, the transmitter 70, monitor 80 and/or the automobile electronics 90 may communicate the sensor data to a central location 100. These locations may be operational centers where an operator may assist the patient with any medical emergencies based on high and/or low blood glucose readings. In further embodiments, the transmitter, monitor and/or automobile electronics may transmit the sensor data to a specific medical center allowing selected physicians to monitor the sensor data, provide suggestions on treatment regimens, and/or assist the patient with overcoming dangerously high or low blood glucose levels. In still additional embodiments, the On-Star® technology may be utilized to contact the appropriate protective services based on dangerously high and/or low blood glucose readings. Paramedics, police and/or fire departments may be contacted depending on the severity of the situation.

In further embodiments, for vehicles not equipped with On-Star® type technology, the transmitter, monitor and/or the automobile electronics may transmit the data to a central location using standardized wireless protocols including Wi-Fi®, GPS satellite, cellular network, or the like.

While the description above refers to particular embodiments of the present invention, it will be understood that many modifications may be made without departing from the spirit thereof. The accompanying claims are intended to cover such modifications as would fall within the true scope and spirit of the present invention.

The presently disclosed embodiments are therefore to be considered in all respects as illustrative and not restrictive, the scope of the invention being indicated by the appended claims, rather than the foregoing description, and all changes which come within the meaning and range of equivalency of the claims are therefore intended to be embraced therein.

What is claimed is:

1. An automobile monitoring system for monitoring patient body characteristics, the automobile monitoring system comprising:
at least one sensor to monitor at least one patient body characteristic;
at least one transmitter operatively coupled to the at least one sensor to communicate sensor data;
at least one monitor operatively coupled to the at least one transmitter to receive the sensor data;
automobile electronics operatively coupled to the at least one transmitter to receive sensor data,
wherein the at least one monitor and the automobile electronics display the sensor data to the patient.

2. The automobile monitoring system of claim 1, wherein the at least one patient body characteristic is blood glucose.

3. The automobile monitoring system of claim 1, wherein the at least one transmitter communicates with the at least one monitor and the automobile electronics using at least one wireless protocol.

4. The automobile monitoring system of claim 3, wherein the at least one wireless protocol is selected from the list of Bluetooth, infrared, radio frequency, 802.11a, 802.11b, or 802.11g.

5. The automobile monitoring system of claim 1, wherein the automobile electronics include at least one of a GPS navigation system, a DVD entertainment system, an on-system computer, or a stereo system.

6. The automobile monitoring system of claim 1, wherein the automobile electronics include default maximum and minimum thresholds for the at least one patient body characteristic.

7. The automobile monitoring system of claim 6, wherein the automobile electronics prevent ignition of the automobile when the sensor data is above the maximum threshold or below the minimum threshold.

8. The automobile monitoring system of claim 6, wherein the automobile electronics display at least one warning when the sensor data is above the maximum threshold or below the minimum threshold.

9. The automobile monitoring system of claim 6, wherein the automobile electronics sound at least one alarm when the sensor data is above the maximum threshold or below the minimum threshold.

10. The automobile monitoring system of claim 9, wherein the at least one alarm provides at least one of audio, visual or tactile indications.

11. A method for monitoring patient body characteristics in an automobile, the method comprising the steps of:
installing at least one sensor in the body of a patient to monitor at least one patient body characteristic, wherein the sensor is coupled to at least one transmitter to communicate sensor data;
sending the sensor data to at least one monitor operatively coupled to the at least one transmitter;
sending the sensor data to automobile electronics operatively coupled to the at least one transmitter, and
displaying the sensor data on the at least one monitor and the automobile electronics.

12. The method of claim 11, wherein the at least one patient body characteristic is blood glucose.

13. The method of claim 11, wherein the at least one transmitter communicates with the at least one monitor and the automobile electronics using at least one wireless protocol.

14. The method of claim 13, wherein the at least one wireless protocol is selected from the list of Bluetooth, infrared, radio frequency, 802.11a, 802.11b, or 802.11g.

15. The method of claim 11, wherein the automobile electronics include at least one of a GPS navigation system, a DVD entertainment system, an on-system computer, or a stereo system.

16. The method of claim 11, wherein the automobile electronics include default maximum and minimum thresholds for the at least one patient body characteristic.

17. The method of claim 16, wherein the automobile electronics prevent ignition of the automobile when the sensor data is above the maximum threshold or below the minimum threshold.

18. The method of claim 16, wherein the automobile electronics display at least one warning when the sensor data is above the maximum threshold or below the minimum threshold.

19. The method of claim 16, wherein the automobile electronics sound at least one alarm when the sensor data is above the maximum threshold or below the minimum threshold.

20. The method of claim 19, wherein the at least one alarm provides at least one of audio, visual or tactile indications.
